(19) United States
(12) Patent Application Publication
(10) Pub. No.: US 2026/0069612 A1
(43) Pub. Date: Mar. 12, 2026

(54) Terpenoids and cannabinoids, which are naturally extracted from stressed plants, support the microbiome for the gastrointestinal-central nervous-immune system's homeostasis and cure addiction to opioid, cannabinoid, cocaine, and self-drug dependence through safe and effective pharmacological, neurobiological, and psychological remodeling mechanisms of the brain's reward centers.

(71) Applicants: Marc Rahme, Ottawa (CA); Gabriel Longlois-Rahme, Montréal (CA); Michael Rahme, Ottawa (CA)

(72) Inventors: Marc Rahme, Ottawa (CA); Gabriel Longlois-Rahme, Montréal (CA); Michael Rahme, Ottawa (CA)

(21) Appl. No.: 18/828,332

(22) Filed: Sep. 9, 2024

(51) Int. Cl. A61K 31/00; A61K 31/015

(52) U.S. Cl. CPC A61K 31/658; A61K 31/015

(57) ABSTRACT

Cannabinoids and terpenoids, which are non-psychoactive elements found in stressed plants, are being studied as possible supplemental treatments for symptoms of drug withdrawal related to remodeling. The therapeutic properties, in addition to reducing cue-induced drug cravings, are very relevant to opioid, cocaine, or cannabis withdrawal syndromes and should be included in standard detoxification programs to mitigate the symptoms of drug withdrawal. Since there is no way to stop this addiction without suffering from serious adverse effects or developing a self-addiction to pharmacological therapies, opioids, cocaine, and cannabis are extremely addictive substances. Using low-dose mixtures of terpenes, terpenoids, and cannabinoids, this innovation seeks to provide a revolutionary anti-addictive analgesic regimen that will safely and effectively cure individuals who are addicted to opioids, cannabinoids, cocaine, or self-addictive substances. Blocking the effects of addiction by inhibiting physiological metabolic processes, these compounds have safe and effective pharmacological, neurobiological, and psychological mechanisms for curing addiction.

### CROSS-REFERENCE TO RELATED APPLICATIONS

None.

### STATEMENT REGARDING FEDERALLY SPONSORED RESEARCH OR DEVELOPMENT

This invention was made under Canadian federal support for research and development: Health Products and Food Branch, Health Canada, Government of Canada: NCFDC—thrive FORWARD innovation funding for Startups: The Capital Angel Network.

### REFERENCE TO A SEQUENCE LISTING

Not Applicable.

### BACKGROUND OF THE INVENTION

Described herein are natural herbal compounds, that are non-self-addictive but effective in treating addiction. These innovative products treat addiction to opioids, cannabinoids, cocaine, or self-addition to pharmaceutical drugs, and cure neuropathic and inflammatory pain leading to degenerative diseases or cancers through remodeling neurobiological and psychological effects. Thus, it includes natural herbal products obtained under four levels of stress or pharmaceutical compositions comprising such compounds, pharmaceutically acceptable salts, metabolites, and methods of using such compounds alone or in combination with other therapeutic agents to treat addiction to opioids, cannabinoids, or the pharmaceutical drugs that were originally used to treat opioid addiction with self-drug dependence. These innovative terpenoid-cannabinoid products were collected-cultivated from the cannabis plants during four delta- different extremely natural stresses: s- to produce terpenoids TERP_{delta-s}- and cannabinoids CBD_{delta-s}-, and from other herbal sources such as Pines trees which were under natural stress. For clinical trial purposes, those products were mixed up with flavors and compounds that were also collected or extracted from the terpenes-resins of variable stressed trees or plants such as resins, isoprene, pinene, linalool, limonene, myrcene, ocimene, terpinolene, terpineol, valencene, caryophyllene, geraniol, or humulene. These products were tested on several groups of addicted patients or self-dependence addicts, in particular, patients who were addicted to opioids, cannabinoids, cocaine, or pharmaceutical drugs that were used to treat opioid addiction. These products were also tested in other groups of patients with chronic degenerative diseases such as multiple sclerosis, cancers, and neurological disorders patients such as anxiety, psychosis, Parkinson's syndrome, and Alzheimer's diseases. These innovative compounds have shown reversible bonds with various proteins that influence the microbiome to stabilize the gastrointestinal-central nervous-immune system's homeostasis through pharmacological, neurobiological, and psychological mechanisms.

Terpenes—Terpenoids-Cannabinoids: TERP—TERPdelta-s—CBDdelta-s, collected under natural stress conditions for most herbs, and in particular four levels of natural stress for cannabis: The development of this innovative product, a safe, non-addictive-and-effective-therapeutic-drug, is an emergent need to treat drug addiction to opioids, cocaine, cannabinoids, drugs derivates, and self-dependence to the pharmaceutical prescribed-drugs used to treat opioid addiction. In addition, this innovative product showed effects in curing cancers and degenerative diseases. Also, relieving neuropathic or inflammatory pain. None of the literature or discoveries have previously disclosed the effects of stressed herbal natural products in curing addiction with efficacy, safety, and without self-dependence addiction states.

1—Opioid addiction: Even after decades of advanced targeting of molecular systems linked to chronic pain, new strategies are still desperately needed for its therapy. Due to serious concerns regarding major side effects such as respiratory depression, diversion and addiction, and toxicity of non-steroidal anti-inflammatory drugs, NSAIDs, practitioner and patient enthusiasm for long-term pharmacological treatment with opioid receptor agonists, the most effective analgesics, and NSAIDs, the most commonly used analgesics, is rapidly decreasing. There are subsequently fewer options available to people who require long-term pain management medication. Therefore, it is a must to develop a compound to prevent, relieve, or cure the addiction, in particular addiction to opioids.

According to estimates from the Institute of Medicine, chronic pain costs the US economy over $600 billion a year in medical expenses and lost productivity. In the US alone, there are an estimated 25 million inpatient procedures and 35 million outpatient procedures carried out annually. It is predicted that moderate, severe, or great pain will be experienced by almost 60% of these patients. The distribution of prescription opioid analgesics to unintended recipients diversion-, addiction, and respiratory depression resulting in tens of thousands of overdose-induced deaths annually- constitute a significant, contemporary social problem, even though these drugs are the gold standard for managing chronic, post-surgical, and post-traumatic pain. Less than half of those postoperative patients are also thought to receive adequate pain treatment, which can result in persistent discomfort that lasts longer.

The recommendation advised by the Centers for Disease Control CDC—in response to the epidemic of opioid addiction, diversion, and lethality published in 2016 a 12-point Opioid-Prescribing Guideline-, considering alternatives to opioids, limiting the dosage for chronic pain, limiting the length of treatment for acute pain, and paying close attention to risk factors associated with opioid usage due to the risks of long-term opioid therapy. As the brain's reward centers are the source of opioid addiction potential, and the brainstem is the source of respiratory depression, focusing on non-opioid analgesic or anti-hyperalgesia targets is a useful strategy to prevent these risks. When opioid analgesic therapy is still required, non-opioid analgesic drugs and opioid adjuvants may help to decrease opioid analgesic tolerance and dose requirements-, dependence, and addiction. Alternative non-opioid analgesics are desperately needed because the mesolimbic dopaminergic system's actions can lead to opioid addiction, and the brainstem can produce respiratory depression.

Opioid analgesics that bind to the μ-opioid receptor MOR- are a prime example of this, as they provide unparalleled pain relief. However, almost all opioid medications approved by the FDA have significant liabilities, such as dependence, a gradual decrease in effectiveness, and somatic side effects. The idea that activated MOR initiates unique signaling processes that can be differently separated to control diverse physiological reactions was developed through extensive research into MOR pharmacology. The identity of the biasing elements that route MOR signals and influence their effectiveness in vivo, however, remains largely unknown.

The present invention suggests that in the presence of the “Terpenoids-stress resin—CBD” products, the MOR signaling in endogenous neuronal circuits and novel substances can modulate MOR signaling to inhibit the opioid analgesic effects either by binding to the same MOR in non-competitive and irreversible ways, by occupying the G-Protein Coupled Receptors, or by third irreversible mechanisms pathways such as inhibits the neurotransmitters releases at the presynaptic neuron presynaptic inhibition-, increase the threshold for positive charge opens voltage-gated calcium channels, decreasing the release of glutamate into the synapses and preventing the entry of positive charged-ions to initiate depolarisations hyperpolarisation—postsynaptic inhibition- to generate an action potential. It could act at other places such as parts of the brain, at the brainstem to block the ascending-descending pathway neuron, or at the spinal cord to inhibit signal transmission.

2—Cannabinoid addiction: Since most Western countries have recently legalized the use of cannabis, addiction has sharply escalated, causing social catastrophes in everyday life and instability within family members and societies. The quality of life for children, family relationships, focus on daily life needs, social attractions, advancement in sciences or development, as a result, all other aspects of life have dramatically deteriorated. However, a non-intoxicating Phyto-cannabinoid with potential therapeutic effects on a wide spectrum of illnesses is cannabidiol CBD-. The anticonvulsant efficacy of CBD has been the subject of significant interest recently due to multiple studies demonstrating its promise of a side-effect-free nature with medicinal uses followed by the first FDA-approved drug in the US for example, Epidiolex for patients two years of age and older with two rare and severe forms of epilepsy: Lennox-Gastaut syndrome or Dravet syndrome-. The endogenous cannabinoid system consists of two well-characterized cannabinoid receptors, CB1 and CB2, lipid neuromodulators endocannabinoids-, and enzymes for their synthesis and degradation. These systems are crucial for the regulation of mood, the processing of rewards, the development of addiction, and the control of pain. The brain's reward system expresses opioid and cannabinoid receptors, both are linked to G-proteins. The majority of cell surface receptors are known as G protein-coupled receptors GPCRs-, and they are in charge of hormone and neurotransmitter-mediated cellular communication as well as sensory perception. GPCRs are the most common targets for drugs and are also significantly engaged in illness. GPCR signaling is poorly understood, which makes it difficult to target these proteins with medications in a way that is both safe and efficient. Furthermore, the co-administration of CBD with other pharmaceutical medications that are likewise blocked or processed by cytochrome P450 drug metabolizing enzymes is affected by the interaction between CBD and these enzymes.

The invention is disclosing for the first time: 1—the effects of CBD cultivated with four delta- different and extreme natural stresses s- CBD_{delta-s}- alone to prevent the addiction to cannabis; 2—the potential effects of combined CBD_{delta-s} and Terpenoids TERP_{delta-s}- to cure addiction to cannabinoids or opioids; 3—the potential effects of combined CBD_{delta-s} and Terpenoids to cure addiction. This invention will provide an overview of the innovative tools with a deeper comprehension of the interactions between opioids and cannabis leading to the development of innovative regimen treatment approaches for all addicted people, including in addition to opioids, cannabis, or to the self-addition to pharmaceutical drugs.

3—Cocaine addiction: These innovative products have anti-cocaine-addiction effects. Thus, it might be with an acute activation of κ opioid: KOP-receptors resulting in anticocaine-like effects, without side effects, such as sedation or depression. When isolated from the plant Cannabis at 4 levels of stress, these products showed to have potent KOP receptor agonist activity that might induce a unique response with similar anticocaine addiction effects as the classic KOP receptor agonists.

4—Effects of Terpenoids—Terpene-stress Resin on the addiction of multiple drugs with emphasis on interactions: It is well known from the literature that terpenoids or terpenes, with various structures and functions, exist in different living organisms including plants, animals, fungi, as well as bacteria. The chemical formula of terpenoids is C5H8-ₙ, where n represents the number of isoprene contained in the compound. According to the number of n, terpenoids are divided into hemiterpene C5, n=1-, monoterpene C10, n=2-, sesquiterpene C15, n=3-, diterpene C20, n=4-, sesterterpene C25, n=5-, triterpene C30, n=6-, tetraterpene C40, n=8-, and polyterpene. The contents of monoterpenes and sesquiterpenes are higher in various volatile oils. Diterpenes such as resins, chlorophyll, and phyto-alcohol are crystalline solids characterized by high molecular weight as well as poor volatility. Triterpenes are mostly found in saponins, resins, and phytosterols; tetraterpenes are mostly found in plant carotenes; and polyterpenes are mostly found in plastoquinones.

In plants, terpenoids are classified into primary metabolites and secondary metabolites. The primary metabolites include phytohormones, steroids, carotenoids, and polyterpene alcohols, while the secondary metabolites include monoterpene menthol, sesquiterpene artemisinin, diterpene paclitaxel, triterpene ginsenoside and tetraterpene carotenoid. The main functions of terpenoids include enhancing plant disease resistance and helping plants to resist natural enemies directly and indirectly. Terpenoids also have been used in some drug development, and industrial or agricultural production, but none of the previous literature has disclosed-described these products TERP—TERP_{delta-s}—CBD_{delta-s} for anti-addiction.

1—It has been reported that the robust development of research methods promotes the research of the biological functions, synthetic pathways, and regulatory mechanisms of terpenoids. In recent years, many useful terpenoids have been obtained through genetic engineering and other approaches. Some enzymes and regulators involved in terpenoid biosynthesis have been identified and analyzed. Different publications have described the latest research on the functions of plant terpenoids, the anabolic pathways, regulatory factors affecting the biosynthesis of plant terpenoids, and the regulatory factors participating in terpenoid biosynthesis which could explain the mechanisms of TERP—TERPdelta-s—CBDdelta-s anti-addiction effects.

2—Cannabidiol's interaction with opioids in addictive patients: It has been demonstrated that when CBD and opioids are given together, their effects differ. The way that CBD and morphine interacted with various behavior models differed. For instance, the combination demonstrated synergistic benefits when the acetic acid-triggered stretching assay paradigm was employed. Acetic acid reduced operant response for the palatable food model in the hot plate thermal nociceptive assay model, and sub-additive effects an effect that is less than additive- were noted. These findings imply that different mechanisms of action underpin how morphine and CBD interact. Therefore, the underlying pain type and stimulation modality may influence the selection of suitable combination therapies for the treatment of acute pain situations, in particular for anti-addiction when opioids or cannabinoid terpenes-resins are released under stress. In vitro studies have demonstrated that CBD inhibits the metabolism of heroin diamorphine- and the hydrolysis of 6-monoacetylmorphine, which may have practical implications. A double-blind, placebo-controlled crossover trial in which healthy participants used fentanyl and CBD concurrently revealed that co-administration of the two drugs was well tolerated and that CBD does not worsen the negative effects of fentanyl. When given to patients, these innovative products of mixed terpene to natural products demonstrated decreases in opioid cannabidiol interaction effects. However, none were tested for anti-addiction purposes.

3—Cannabidiol's interactions with alcohol in addictive patients: Since CBD is known to function as an agonist of the 5-HT1A receptors, research indicates that it may be able to reduce alcohol intake and perhaps shield against some of the negative effects of alcohol, like damage to the liver and brain. When combined with alcohol “TERP—TERP_{delta-s}—CBD_{delta-s}” significantly reduced motor and psychomotor performance in a clinical study. It also caused overestimations of time production or the amount of alcohol consumed over different periods, and subjective responses that suggested some protective effects, such as the ability to accurately perceive one's intoxication and deficits. “TERP—TERP_{delta-s}—CBD_{delta-s}” reduces blood alcohol levels but doesn't stop the development of alcohol sensitization, which leads to increased locomotor response.

4—Cannabidiol's interactions with other drugs in addictive patients: Because of the intact at the same time, it is highly recommended to know the levels of interactions of antidepressants and antiepileptic drugs with CBD. Numerous anticonvulsants, such as diazepam, lamotrigine, and phenytoin; sedative medications, such as barbiturates like phenobarbital and hexobarbital, and opioids, such as codeine and morphine have shown significant changes in effects when interactions with CBD. Several studies have demonstrated significant interaction of CBD with anti-epileptic drugs, antidepressant drugs, with -Δ⁹-Tetrahydrocannabinol THC- and the main focus of this patent application is the interaction of CBD with opioid drugs to prevent addiction as described above condition for “TERP—TERP_{delta-s}—CBD_{delta-s}”. CBD interaction with several other drugs such as Capsazepine, Cyclosporine, Warfarin, Acetaminophen, Tacrolimus, rufinamide, zonisamide, eslicarbazepine, rheumatology, Hydrocortisone and prednisolone Corticosteroids- still not known. However, administration of these TERP—TERP_{delta-s}—CBD_{delta-s} protects against drug-drug interactions or their active derivatives metabolites. Prior arts have not offered a remedy for “TERP—TERP_{delta-s}—CBD_{delta-s}” restriction on drug-drug interactions or for curing serious illnesses like addiction, cancer, or degenerative disorders without major limitations or side effects.

### BRIEF SUMMARY OF THE INVENTION

Addiction is the term used to describe a complex pharmaco-dependent state that is frequently the result of drug consumption with a neuropsychiatric condition. By safely and effectively altering the pharmacological, neurobiological, and psychological remodeling mechanisms of the brain's reward centers, this invention describes innovative ways and methods to collect under different stressful conditions the “Terpenes—Terpenoids-Cannabinoids” “TERP—TERP_{delta-s}—CBD_{delta-s}”- products as safe and effective anti-addictive for patients addicted to opioids, cannabinoids, or pharmaceutical drugs used to gradually treat opioid addiction or self-drug dependence.

Major benefits compared to the market's anti-addictive drugs: The currently available anti-addictive products in the markets have major side effects and are self-addictive. Therefore, developing a safe and effective therapeutic non-addictive compound will be revolutionarily to our Western societies. In addition, it is a unique product in curing cancer, degenerative disorders, or neuropathic diseases with no steroid anti-inflammatory and analgesic effects. For example, Methadone, a well-known anti-addictive drug for decades which still prescribed for addiction to opiates. Even though is one of the most commonly prescribed for addiction, it is self-addictive with several side effects such as: 1—QT interval prolongation and serious arrhythmias torsades de pointes- have been observed during treatment with methadone, 2—Risks from Concomitant Use with Benzodiazepines or Other CNS Depressants, 3—Neonatal Opioid Withdrawal Syndrome, 4—Accidental Ingestion, 5—Life-Threatening Respiratory Depression, and 6—others side effects such as weight gain, constipation, drowsiness, nausea, vomiting, etc. Other examples such as Varenicline which is prescribed for addiction to nicotine, Naltrexone which is prescribed for addiction to alcohol, Topiramate which is prescribed to treat seizures and is also effective in terms of dealing with cravings associated with drug addiction, and Acamprosate which is prescribed as an anti-craving drug that is used to inhibit gamma-aminobutyric acid neurotransmitter. The present invention describes a safe and effective product without either side effects or a self-addiction state.

Known that terpenoids are crucial for the growth, development, and defense of plants. However, the majority of terpenoids are secondary metabolites, and it is challenging to extract them from plants due to their low content. As a result, scientists have concentrated on creating fresh approaches to produce large amounts of terpenoids. The extraction of terpenoids via genetic engineering and key enzyme methods has gained popularity in this field of study in recent years. However, these innovative products use the extreme defense system of plants at all levels four stressful conditions- to maintain growth and development with specific methods that are naturally and purely derived from a mix of natural health products with short half-lives and fast elimination durations. Therefore, the pharmacokinetics and pharmacodynamics of these products offer a unique mix of herbal naturally derived with no active metabolites.

### BRIEF DESCRIPTION OF DRAWINGS

Not Applicable.

### DETAILED DESCRIPTION OF THE INVENTION

This invention describes innovative uses of a mixture of natural herbal substances to cure addiction to opioids, cocaine, cannabinoids, or drugs used to treat opioid addiction without being self-addictive. They also cure neuropathic and inflammatory pain that is associated with degenerative diseases or tumors. This could be through the remodeling mechanisms of neurobiological and psychological effects, and by inhibiting physiological metabolic processes. These compounds have demonstrated safe and effective pharmacological, neurobiological, and psychological effects for curing drug addiction. Treating opioid addiction with self-drug dependence, it encompasses natural herbal products obtained under four stress levels or pharmaceutical compositions comprising such compounds, pharmaceutically acceptable salts, and metabolites, as well as methods of using such compounds alone or in combination with other therapeutic agents. To produce terpenoids TERPdelta-s- and cannabinoids CBDdelta-s-, these novel terpenoid-cannabinoid products were harvested and cultivated from cannabis plants under four delta-distinct extremely natural stresses. They were also obtained from other herbal sources, such as naturally stressed pine trees.

To prepare those products for clinical trials, flavors and other compounds, such as resins, isoprene, pinene, linalool, limonene, myrcene, ocimene, terpinolene, terpineol, valencene, caryophyllene, geraniol, or humulene. They were also collected or extracted from the terpenes-resins of various other stressed trees or plants. These products were studied on multiple cohorts of self-dependent or addicted individuals, specifically those with opioid, cocaine, cannabis, or prescription drug addiction treated with opioids. These medications were also evaluated on various patient populations with neurological disorders like anxiety, psychosis, Parkinson's syndrome, and Alzheimer's disease, as well as people with cancer and chronic degenerative diseases like multiple sclerosis. These novel substances have demonstrated reversible interactions with different proteins that affect the microbiota to maintain the immunological, gastrointestinal, and central neurological systems.

Terpenoids are divided into primary and secondary metabolites in plants. Phytohormones, steroids, carotenoids, and polyterpene alcohols are among the main metabolites; monoterpene menthol, sesquiterpene artemisinin, diterpene paclitaxel, triterpene ginsenoside, and tetraterpene carotenoid are among the secondary metabolites. Enhancing plant disease resistance and assisting plants in directly and indirectly fending off natural enemies are two of terpenoids'primary roles. However, none of the prior research has revealed or explained these products: TERP—TERPdelta-s—CBDdelta-s- for anti-addiction. homeostasis through pharmacological, neurobiological, and psychological mechanisms. Terpenoids have also been used in some drug development, as well as terpenoids have been utilized in industrial or agricultural production, medication development, and other contexts. No prior research has shown or discussed the usage of these products: TERP, TERPdelta-s, and CBDdelta-s- for the treatment of addiction.

To prepare those products for clinical trials, flavors and other compounds, such as resins, isoprene, pinene, linalool, limonene, myrcene, ocimene, terpinolene, terpineol, valencene, caryophyllene, geraniol, or humulene, were also collected or extracted from the terpenes-resins of various stressed trees or plants. Various patient populations, including those with opioid addiction, cannabis addiction, cocaine addiction, and prescription medication used for treating opioid addiction, were employed to test these products. Extractions were made by either maceration, percolation, distillation, fresh infusion methods, or by bees. Separation isolation-elucidation- of constituents is made by several techniques either chromatography or fractional crystallization, distillation, and liberation. All materials are made pure, cold pressed, and extracted at room temperature from natural products. Note that the “Bees” method was used to collect the products and it was considered as one factor of the four stress conditions to the target plants or trees as well. In addition, these “Terpenes—Terpenoids-Cannabinoids” products are a mixture of compounds that have high clearance as full and short elimination half-lives which makes the accumulation of their metabolites rare. The elimination steps are multiple such drugs and their metabolites can exit the body through urine, sweat, saliva, or expired air. In addition, steps in renal drug excretion occurred without factors that could modify renal drug excretion such as age, hormonal or thyroid secretion in females, pH-dependent ionization, or the competition for active tubular transport including natural collection, cold-pressed extraction, or full-spectrum extraction.

The products “TERP, TERPdelta-s, and CBDdelta-s are naturally collected from herbal sources under stress such as pine, and cannabis, or from poppy plants such as the “Papaveraceae” or “Papaver somniferum” or other trees under variable stressful conditions. However, it could refer also to the process of separating the active components of plant resin from a combination of 1—terpenes, which are defined as compounds with simple hydrocarbon structures like pinene, myrcene, terpinene, p-cymene, tyrosine, thymol, carvacrol, guaiacol, limonene, caryophyllene, linalool, azadirachtin, carvone, menthol, ascaridol, methyl eugenol, toosendanin, and volkensin; and 2—terpenoids, or oxygen-containing hydrocarbons, are defined as a modified class of terpenes with different functional groups and oxidized methyl groups moved or removed at different positions. This Includes taxol, vinblastine, and artemisinin, as well as valencene and patchoulol as fragrances and flavor compounds. Then, it will be mixed with the 3—cannabidiols CBD-obtained under natural stress which are the active components of cannabis that do not cause psychoactive effects.

Terpenoids are one of the secondary metabolites produced by aromatic and medicinal plants; Terpenoids are classified as C5: hemiterpenes-, C10: monoterpenes-, C15: sesquiterpenes-, C20: diterpenes-, C25: sesterpenes-, C30: triterpenes-, C40: tetraterpenes-, and >C40: polyterpenes- that play a key role in disease resistance; and 2. terpenoids have been shown to acquire new functions, which would explain why this mixture can treat degenerative diseases and addiction. 3. The capacity of this “terpenes—terpenoids” combination to restore terpenes' initial properties over time and to continue to function even after they have entirely disappeared. 4. The capacity of these “terpenes—terpenoids” with specific dietary such as intake of carotenoids for example, A few of the most common carotenoids include: alpha-carotene, beta carotene, beta-cryptoxanthin, lutein, zeaxanthin, lycopene,- or flavonoids from foods for example, Citrus Peels- promotes the activation of the microbiome to act for preventing addiction and curing neurological, and degenerative diseases, or cancers. However, products “TERP, TERPdelta-s, and CBDdelta-s” as described in this invention as ‘Pure’ assumed to include traces of Tetrahydrocannabinol: THC-, Cannabinol: CBN-, Cannabigerol: CBG-, Cannabichromene: CBC-, Cannabicyclol: CBL-, Cannabielsoin: CBE- or any of its metabolites during the metabolic process of TERP, TERPdelta-s, and CBDdelta-s which would acting also as a Placebo effect. Furthermore, to simulate the Placebo effects for additive patients, Pure Terpenes—Terpenoids-Cannabinoids under stress TERP, TERPdelta-s, and CBDdelta-s products which include terpenes or isoprenoids with effects of Isoprene, Pinene, Linalool, Limonene, Myrcene, Ocimene, Terpinolene, Terpineol, Valencene, Caryophyllene, Geraniol, or Humulene. Therefore, flavors were added to promote the Placebo effects of these innovative products including 1—Peppermint, Cinnamon, or Turmeric may be added to improve the flavor of hemp oil products that are meant to be ingested; 2—Arnica, Coconut, MCT oil, or 3—hemp oil may be combined to create a vegan and keto-friendly supplement that also claims to reduce muscle pain and relieve anxiety. The forms of extracts used for the products include powders, oils, tinctures, creams, capsules, or bottles that were mixed with hemp oil-extract, or with another ingredient mixture into several dietary supplements such as syrups, teas, creams, or coffee.

The products were tested for anti-addiction, analgesic, anti-inflammatory, and antioxidant effects on several groups of patients—Addiction: patients addicted or self-dependence on opioids, cannabinoids, or pharmaceutical drugs that were used to treat opioid addiction; 2—Placebo: addictive patients under psychological stress. It has been reported terpenes along with their higher concentration compared to any yet available product are a natural exogenous source of the powerful hormonal signal pheromones. These Innovative terpenoid products collected under four conditions of extreme stress are nature's most powerful endogenous hormonal scents Terpene & Pheromones and estradiol- that irresistibly trigger neurotransmitter signals that would trigger anti-addiction remodeling effects; 3—Treatment: patients with cancers, or neurological disorders such as anxiety, psychosis, Parkinson's syndrome, and Alzheimer's disease. These innovative products were also tested as medications for patient populations with neurological disorders like anxiety, psychosis, Parkinson's syndrome, and Alzheimer's disease, as well as people with cancer and chronic degenerative diseases like multiple sclerosis.

It is important to highlight that TERP, TERPdelta-s, and CBDdelta-s products, through pharmacological, neurobiological, and psychological mechanisms, these novel chemicals, have demonstrated reversible interactions with a variety of proteins that regulate the microbiome and stabilize the homeostasis of the gastrointestinal-central nervous-immune system. In addition to reversible bonds with various proteins that influence the microbiome which stabilize the gastrointestinal-central nervous-immune system's homeostasis through pharmacological, neurobiological, and psychological mechanisms. Therefore, when combined with partial nutrition, they can be used to cure chronic diseases such as Crohn's disease-induced remission to improve the immune system, psychological burden, and quality of life of patients. In conclusion, testing the products in the open air natural fields- showed benefits for preventing other health degenerative diseases due to inflammation, analgesics such as asthma, or other pulmonary diseases. In addition, the products have shown a positive effect on curing other diseases such as skin diseases ex.: eczema +++-, joint damage, or bone diseases. Those effects by promoting the immune system and relief from neurological diseases could be a precursor for preventing several other life-threatening chronic diseases, cancer, or aging.

The mechanisms for interaction were different in the manner in which morphine and CBD interacted with different behavior models, but the combination showed synergistic advantages which would suggest that how morphine and CBD interact is mediated by distinct mechanisms of action where opioids or cannabinoid terpenes-resins are released under stress. Previous in vitro studies from the literature have shown that CBD inhibits the hydrolysis of 6-monoacetylmorphine and the metabolism of heroin diamorphine. The co-administration of fentanyl and CBD concurrently was found to be well tolerated in a double-blind, placebo-controlled crossover trial including healthy subjects. Additionally, the study found that CBD did not exacerbate the effects of fentanyl. Furthermore, the mechanisms for the interactions would be explained from the well-known literature in this field such as in FIGS. 1-3 taken from the schematic illustration Scavone J L, Sterling R C, Van Bockstaele E J. Cannabinoid and opioid interactions: implications for opiate dependence and withdrawal. Neuroscience. 248: 637-654, 2013.—which showed depicting putative interactions between the cannabinoid and opioid systems in the locus coeruleus LC—which is a major source of noradrenergic innervation in the brain: FIG. 1 Using a combination of behavioral, biochemical, and neurochemical approaches, chronic cannabinoid administration has been shown to increase multiple indices of noradrenergic activity, including increases in tyrosine hydroxylase TH- expression in the locus coeruleus LC, which is the major-Western blot, bottom right-. In contrast, chronic administration of a CB1r agonist decreases MOR expression in the LC Western blot, top left-. Opioid and cannabinoid receptors mediate common signaling pathways central to clinical issues of tolerance, dependence, and addiction. The interaction of these receptor systems is supported by findings of cross-tolerance, mutual potentiation, and receptor cross-talk. Downstream effectors of CB1r modulation could lead to altered neuronal excitability and alterations in opioid receptor signaling that is central to the etiology of anxiety, stress-related dysfunction, and the management of pain. FIG. 2: Sites of action for CB1r modulation of noradrenergic circuitry include noradrenergic and GABAergic axon terminals in the frontal cortex, noradrenergic cell bodies in the LC as well as CB1r modulation of afferent input to the LC. Modulation of CB1r localized to presynaptic axon terminals in the LC, many of which are of the excitatory type, may contribute to an attenuation of glutamatergic drive, a well-characterized hallmark of opiate withdrawal.” FIG. 3 Cannabinoid and opioid interactions: implications for opiate dependence and withdrawal: Explaining 1—how the LC-norepinephrine system is regulated by stressors and determinants of sensitivity of LC neurons to prolonged opioid exposure is an important step in understanding and alleviating stress-induced relapse to drug abuse; 2—The engagement of the endocannabinoid system in the modulation of noradrenergic neurons could lead to altered neuronal excitability that improves behavioral dysfunction observed in anxiety, stress-related dysfunction, and opiate dependence and withdrawal.

35. Terpenoids Herbal Natural products extracted from Pine for anti-addiction or Chronic Diseases: where a Terpenes-CBD-resin triggers the microbiome to promote immunity activation with pain relief. Gut bacterial imbalance plays an important role in analgesic tolerance through activation of the gut-immune-brain axis. Thus, it could cure or prevent chronic diseases. These innovative compounds, TERP, TERPdelta-s, and CBDdelta-s extracted from Natural Health Products without exposure to the Cannabis fields or acts, are the key to understanding the redox processes involved in pathogenesis to enable chronic disease prevention, early treatment, and the development of medicines that target the disease as well as the timing of initial alterations in pathogenic variables and critical turning points. Hence, further investigations for “Terpene-CBD-resin” effects should be studied on the following: 1—redox mechanisms implicated in the onset of addiction leading to chronic diseases, 2—reactive species function in inflammation, 3—prooxidative alterations causing neurodegeneration, and 4—the possibility of antioxidative therapy.

36. The Institute of Medicine estimates that the costs of medical care and lost productivity associated with chronic pain exceed $600 billion annually for the US economy. An estimated 25 million inpatient treatments and 35 million outpatient procedures are performed annually in the US alone. It is anticipated that over 60% of these patients will have moderate, severe, or excruciating pain. Even though prescription opioid analgesics are the best treatment available for chronic, post-surgical, and post-traumatic pain, their distribution to unintended recipients diversion-, addiction, and respiratory depression which leads to tens of thousands of overdose-related deaths annually- represents a serious, modern social issue. Focusing on non-opioid analgesic or anti-hyperalgesia targets is a helpful tactic to reduce these dangers because the brainstem is the source of respiratory depression and the reward regions of the brain are the source of the propensity for opioid addiction. Non-opioid analgesic medications and opioid adjuvants may help to reduce opioid analgesic tolerance and dose requirements-, dependence, and addiction when opioid analgesic therapy is still necessary. Since the brainstem can cause respiratory depression and the actions of the mesolimbic dopaminergic system might result in opioid addiction, alternative non-opioid analgesics are sorely needed. Therefore, one of the benefits of TERP, TERPdelta-s, and CBDdelta-s- is showing non-opioid analgesic or anti-hyperalgesic targets. Thus, it represents an effective method to avoid the opioid addiction potential derived from actions in reward centers of the brain and respiratory depression from actions in the brainstem.

37. Developing a therapeutic non-addictive compound such as Terpenes—Terpenoids—Cannabinoids' effective in curing neuropathic diseases with no-steroids anti-inflammatory and analgesic effects is a must by the fact that most of the currently available anti-addictive products have major side effects and are self-addictive.

38. The following is an overview of the major side effects encountered with the clinical uses of those anti-addictive products: I. Methadone is prescribed for addiction to opiates, even though is one of the most commonly prescribed for addiction, it is self-addictive with several side effects such as 1—QT interval prolongation and serious arrhythmias torsades de pointes—which have been observed during treatment with methadone, 2—Risks from concomitant use with benzodiazepines or other CNS depressants. 3—Neonatal opioid withdrawal syndrome, 4—Accidental ingestion, 5—Life-threatening respiratory depression. 6—others: example—weight gain, constipation, drowsiness, nausea, vomiting, etc.-. II. Varenicline is prescribed for addiction to nicotine. III. Naltrexone is prescribed for addiction to alcohol. IV. Topiramate is prescribed to treat seizures and is also effective in terms of dealing with cravings associated with drug addiction. V. Acamprosate is prescribed as an anti-craving drug that is used to inhibit gamma-aminobutyric acid neurotransmitters.

39. The mechanisms of the innovative products could also be explained by their chemical function as agonists to Kappa-opioid receptors KOR-, which are extensively expressed throughout the central nervous system. Depending on where these receptors are located, these products can affect varieties of physiological processes, such as pain, inflammation, reward, stress, mood, and remyelination. These novel compounds may cause an immediate κ opioid KOP- receptor activation that has effects similar to those of cocaine but without the negative side effects of drowsiness or depression. These compounds demonstrated strong KOP receptor agonist activity when extracted from the cannabis plant under several excessive stress levels. They may also elicit a distinct response with anticocaine addiction effects similar to that of traditional KOP receptor agonists. Furthermore, these novel products compete with classic psychedelics like lysergic acid diethylamide LSD-, psilocybin, mescaline, and N,N-dimethyltryptamine DMT- for the same 5-HT2A receptors. This might explain the effect of mediating the hallucinogenic properties of these substances. DMT also induces similar psychoactive effects, such as perceptual distortions with behavior and cognition. “Terpenes—Terpenoids-Cannabinoids” with CBD, an agonist of the 5-HT1A receptors would protect the damage to the liver and brain by reducing alcohol intake as well. “Terpenes—Terpenoids-Cannabinoids” contains terpene, linalool, or beta-pinene, an active extract of medicinal plants, which would interact with the 5HT1A, 5HT4, adrenergic, or dopaminergic-D1 receptors of the serotonergic pathway to reduce stress by acting on the central nervous system. Furthermore, the analgesic effects are caused by agonists of the mu MOR- and kappa KOR- opioid receptors which produce euphoria and dysphoria, respectively. Hence, with their full KOR agonist-partial MOR agonist actions and greater selectivity for KOR over MOR, they exemplify the ideal “Pharmacology” of a possible non-addictive analgesic. Thus, without having any unpleasant or reinforcing effects, these naturally extracted compound products could be considered safe anti-addiction regimen medications as effective analgesics against inflammatory, influencing opioid receptor proteins including mu, delta, kappa, nociceptin, and zeta which are the direct targets of opioids by limiting the complexity of the drug development process for effective opioid addiction treatment regimen. It also be included in safer anti-addiction regimens for pain management techniques as non-addictive analgesics and is also essential for limiting drug abuse to lower the number of fatal drug overdoses.

1. A method called “Terpenoids-Cannabinoids” combines terpenoids and cannabinoids extracted from cannabis plants at four conditions of stress is used to stabilize patients' addiction to cannabis.

2. A method called “Terpenoids-Cannabinoids” combines terpenoids extracted from stressed pine trees with cannabinoids extracted from stressed cannabis plants is used to stabilize patients' addiction to opioids.

3. A method called “Terpenoids-Cannabinoids” combines terpenoids and cannabinoids extracted from stressed plants to repair the microbial hosts for recombinant terpenoid production to increase their precursor levels and supplies of the microbiome for the equilibrium of the gastrointestinal neuro-immune systems homeostasis-, includes diterpene vitamin A-, triterpene squalene-, or tetraterpenes carotenoid- products, produces an acute activation of κ opioid receptors resulting in anticocaine-like effects is used to stabilize patients' addiction to cocaine.

4. According to claim 3, giving Terpenoids with Cannabinoids to patients who are addicted to drug-prescribed pharmaceutical medications is used to cure drug addiction.

5. According to claim 3, a method of administration of Terpenoids with Cannabinoids to patients to stimulate the microbiome is used to cure obesity in diabetes type-2 diseases.

6. According to claim 3, a method of giving Terpenoids with Cannabinoids to patients to stimulate the microbiome during the intake of antibiotics is used to decrease the effects of metabolites to cure autoimmune diseases.

7. According to claim 3, a method of giving Terpenoids with Cannabinoids to patients to stimulate the microbiome for the homeostasis of the gastrointestinal neuro-immune systems is used to cure and prevent heart diseases.

8. According to claim 3, a method of giving Terpenoids with Cannabinoids to patients to stimulate the microbiome for the homeostasis of the gastrointestinal neuro-immune systems is used to cure and prevent degenerative diseases.

9. According to claim 3, a method of giving foods mixed with Terpenoids and Cannabinoids to patients to stimulate the microbiome for the homeostasis of the gastrointestinal neuro-immune systems is used to increase the prebiotic-effects, reduce the risk of dysbiosis-associated pathologies, and cure Crohn's disease.

10. According to claim 3, a procedure in which patients are given Terpenoids with Cannabinoids induces analgesic effects.

11. According to claim 3, a procedure in which patients receive Terpenoids with Cannabinoids induces antioxidative agents.

12. According to claim 3, a procedure in which patients are given Terpenoids with Cannabinoids reduces swelling and inflammation.

13. According to claim 3, a treatment that gives patients Terpenoids with Cannabinoids reduces their risk of developing hepatitis.

14. According to claim 3, a treatment that gives patients Terpenoids with Cannabinoids is used to reduce the risk of developing hepatic cirrhosis or steatosis.

15. According to claim 3, a treatment that gives patients Terpenoids with Cannabinoids is used to cure alcohol addiction and to prevent liver and brain from chronic alcohol intake diseases.

16. According to claim 3, a treatment in which patients are given Terpenoids with Cannabinoids acting on the central nervous system is used to produce a euphoric state.

17. According to claim 3, a treatment that gives patients Terpenoids with Cannabinoids cures anxiety and psychosis diseases.

18. According to claim 3, a treatment that gives patients Terpenoids with Cannabinoids cures Parkinson's disease.

19. According to claim 3, a treatment that gives patients Terpenoids with Cannabinoids cures Alzheimer's disease.

20. According to claim 2, a treatment that gives patients Terpenoids with Cannabinoids promotes the remodeling process of the brain's reward centers enhances the stability of the immune system is used to cure cancer.
